(19) United States
(12) Patent Application Publication
(10) Pub. No.: US 2023/0248722 A1
(43) Pub. Date: Aug. 10, 2023

(54) CLOFAZIMINE COMPOSITION AND METHOD FOR THE TREATMENT OR PROPHYLAXIS OF VIRAL INFECTIONS

(71) Applicant: MANNKIND CORPORATION, WESTLAKE VILLAGE, CA (US)

(72) Inventors: Thomas Hofmann, Doylestown, PA (US); Stefan Ufer, Raleigh, NC (US); John J. Freeman, JR., New Fairfield, CT (US)

(73) Assignee: MANNKIND CORPORATION, WESTLAKE VILLAGE, CA (US)

(21) Appl. No.: 17/996,706

(22) PCT Filed: Apr. 30, 2021
(86) PCT No.: PCT/US2021/030155

§ 371 Date: Oct. 20, 2022

Related U.S. Application Data
(60) Provisional application No. 63/018,677, filed on May 1, 2020.

(51) Int. Cl. A61K 31/498; A61K 9/00; A61K 47/26; A61K 47/10; A61K 47/12

(52) U.S. Cl. CPC A61K 31/498; A61K 9/0075; A61K 9/0078; A61K 47/26; A61K 47/10; A61K 47/12

(57) ABSTRACT

Disclosed herein are methods of treatment and/or prophylaxis of viral infections. In particular, the method is for the administration by inhalation of a pharmaceutically effective dose of clofazimine, which is provided in the form of a solution, suspension, or as a dry powder, in formulations suitable for inhalation.

### CROSS REFERENCE TO RELATED APPLICATIONS

This application is a national stage application of International Patent Application No. PCT/US2021/030155, filed Apr. 30, 2021, which claims benefit under 35 U.S.C. § 119(e) from U.S. Provisional Patent Application Ser. No. 63/018,677, filed May 1, 2020, the content of which are incorporated herein by reference in their entirety.

### TECHNICAL FIELD

Disclosed herein are compositions and methods for the treatment and prophylaxis of viral infections, including, coronavirus infections such as COVID-19. In particular, the compositions comprise clofazimine in a suspension or dry powders for inhalation, administered by nebulization or oral inhalation.

### BACKGROUND

A viral disease (or viral infection, or infectious disease) occurs when an organism's body is invaded by pathogenic viruses, and infectious virus particles (virions) attach to and enter susceptible cells. The virus replicates within the infected cells using the cell's own mechanisms and new viruses are released from the cells. The virus infects other cells within the organism and continues to replicate until the immune system cannot overcome the viral load/content, or contain the viral infection, and disease condition ensues.

Coronavirus disease 2019 (COVID-19) is an infectious disease caused by SARS-CoV-2, a virus closely related to the SARS virus. The disease is the cause of the 2019-2020 coronavirus outbreak. It is primarily spread between people by small airborne droplets from infected individuals when they breathe or cough. Time from exposure to onset of symptoms is generally between 2 and 14 days. Hand washing, maintaining distance from people who are coughing, and not touching one's face is recommended to prevent the disease and quarantining for 10-14 if one is exposed to someone who has tested positive for the virus. It is also recommended to cover one's nose and mouth with a bent elbow when coughing.

People may have few symptoms or develop fever, cough, and shortness of breath. Cases can progress to pneumonia and multi-organ failure. At present, there is no approved vaccine or specific antiviral treatment, with management involving treatment of symptoms, supportive care, and experimental measures. The case fatality rate is estimated at between 1% and 3%. The World Health Organization (WHO) and U.S. Centers for Disease Control (CDC) recommend those who suspect that they are carrying the virus wear a surgical face mask and seek medical advice by calling a doctor rather than directly visiting a clinic in person. Masks are also recommended for those who are taking care of someone with a suspected infection. Masks have been recommended for the general public for indoor and outdoor activities.

WHO has declared the 2019-20 coronavirus outbreak to be a Public Health Emergency of International Concern (PHEIC). As of 19 Feb. 2020, only Mainland China was listed as an area with known ongoing community spread of the disease. Currently, almost all countries in the world, including, Europe and the United States have been considerably affected, and the disease has been classified as a worldwide pandemic. While there are vaccines developed and used currently in vaccinating millions of people around the world, the vaccines have been approved by regulatory authorities only for emergency used. There are some approved therapies such as Remdesivir to reduced time to viral infection recovery, but none specifically defined approved therapies for COVID-19 treatment in humans to inhibit the virus. Accordingly, there is a need for new methods and compositions for the treatment of viral infections, and improved alternative methods and compositions for use, in particular, in the treatment and inhibition of coronavirus infections.

### SUMMARY

Disclosed herein are methods and compositions for the treatment of viral infections comprising antibiotics, including, clofazimine to be delivered to the lungs via oral inhalation. The method comprises delivering a dose of a composition to a patient having a viral infection, which dose is lower than a corresponding intravenous or oral dose currently administered, thus reducing the incidence of side effects caused by standard therapies with clofazimine. The method is advantageous as it facilitates patient treatment with a dose that is less than oral tablets and can reach its site of action quickly with potentially less toxic side effects at lesser amounts.

In one embodiment, the method comprises administering to a patient in need of treatment a therapeutically effective dose of a clofazimine composition to be delivered to the patient's lungs. The clofazimine composition can be provided to the patient in the form of a neat drug, or a pharmaceutically acceptable derivative, polymorphs of clofazimine, or salt thereof. In some embodiments, the clofazimine composition comprises a pharmaceutically acceptable carrier or excipient. In certain embodiments, the clofazimine composition can comprise a solution, suspension, or a dry powder for inhalation, which can be used with a nebulizer, a metered dose inhaler or a dry powder inhaler.

In another embodiment, there is provided a prophylactic method against viral infections of the lung comprising administering to a subject a composition comprising slow release of clofazimine in the lungs, which increases the residence time of clofazimine in the lungs and provides protection against pulmonary viral infections.

In one embodiment, an improved method for treating pulmonary viral infections is provided, said method comprising, delivering a therapeutically effective amount of a composition of clofazimine, a pharmaceutically acceptable derivative, polymorphs of clofazimine, or a salt of clofazimine, to the patient's lungs with an aerosol, wherein the effective amount of clofazimine, pharmaceutically acceptable derivative, a polymorph of clofazimine, or salt of clofazimine, delivered to the lungs is lower than the therapeutically effective oral dose.

In some embodiments, a method of treatment is disclosed, comprising administering to a subject in need a therapeutic amount of a composition comprising clofazimine and a pharmaceutically acceptable carrier and/or excipient, wherein the viral infection is coronavirus, influenza, ebola, or other viral infection affecting the lung, or a combination thereof.

In a particular embodiment, the method comprises, administering to a subject diagnosed with positive SARS-CoV-2 infection, a therapeutically effective amount of a clofazimine composition comprising clofazimine, a pharmaceutically acceptable derivative, polymorphs of clofazimine, or a salt of clofazimine, including, the hydrochloride salt of clofazimine, including, clofazimine acetate, clofazimine citrate, clofazimine phosphate, clofazimine oxalate, clofazimine sulfate, or combinations thereof, and a pharmaceutically acceptable excipient and/or carrier, to inhibit viral replication.

In another embodiment, a method for treating COVID-19 disease comprising administering to a patient in need thereof, a therapeutically effective amount of a clofazimine composition comprising clofazimine, a pharmaceutically acceptable derivative, or a salt thereof, or combinations thereof, and a pharmaceutically acceptable excipient and/or carrier, to inhibit viral replication and viral disease.

In one embodiment, the method of treatment comprises, administering to a subject a therapeutically effective amount of clofazimine composition, wherein the clofazimine, a pharmaceutically acceptable derivative, or salt thereof, in an amount of about 1 mg to about 30 mg; from about 1 mg to 20 mg; from 1 mg to 10 mg; from about 3 and 8 mg, or from about 2 mg to about 6 mg of clofazimine; derivative or salt thereof per dose in the composition to be delivered to the lungs. In an embodiment, the total amount of powder for inhalation including a pharmaceutically acceptable excipient can comprise up to 50 mg per dosage to be administered, and delivered to the lungs with a dry powder inhaler in one, or more than one inhalations or in a nebulized suspension with a nebulizer in one or more than one breaths. In this and other embodiments, a dry powder clofazimine composition is delivered as a unit dose from a cartridge or a capsule in an aerosolized form using a metered dose inhaler or a dry powder inhaler and can be administered daily.

In some embodiments, the clofazimine is delivered by nebulization from a nebulizer and can comprise a suspension comprising a saline solution or a suspension.

### DETAILED DESCRIPTION

In embodiments disclosed herein there is provided a method for the treatment of viral infections comprising the use of clofazimine inhalation suspension (CIS) or inhalable dry powders as a countermeasure to coronaviruses and in particular, SARS-CoV-2, which causes COVID-19 disease. The clofazimine in the composition will inhibit viral infection and is administered directly to the lungs in a clofazimine suspension, or in dry powder form. CIS is currently in preclinical development for the treatment of both nontuberculous mycobacterial pulmonary disease, and tuberculosis and is currently undergoing first-in-human GLP toxicology studies.

In an exemplary embodiment, a method for treating viral infection comprising administering a composition comprising an antibiotic compound having the formula:

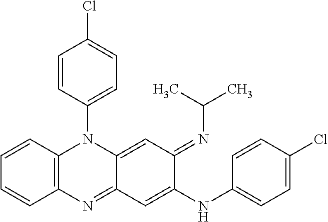 [SMILES: CC(C)/N=c1\cc2n(-c3ccc(Cl)cc3)c3ccccc3nc-2cc1Nc1ccc(Cl)cc1]

and a pharmaceutically acceptable carrier and/or excipient. In an embodiment, pharmaceutically acceptable derivatives, or salts of the compound are also used in the formulation alone or in combinations thereof with the compound, in particular, in compositions for pulmonary inhalation in the treatment of viral lung disease. In one embodiment, the compound is clofazimine, chlofazimine, N,5-bis(4-chlorophenyl)-3-propan-2-yliminophenazin-2-amine, clofazimine, clofaziminum, 3-(p-chloranilino)-10-(p-chlorphenyl)-2,10-dihydro-2-(isopropylimino)-phenazine, or polymorphic forms or polymorphs of clofazimine; including, a triclinic (F I) polymorph, a monoclinic (F II) polymorph, an orthorhombic crystal polymorph (F III) and a high temperature polymorph (F IV). Clofazimine salts include, for example, the hydrochloride salt of clofazimine, clofazimine acetate, clofazimine citrate, clofazimine formate, clofazimine phosphate, clofazimine oxalate, clofazimine sulfate and the like. In embodiments herewith, clofazimine may refer to any form of clofazimine when used alone, or in combination with others in a composition, unless derivatives, salts or polymorphs are specifically recited.

In another example embodiment, the composition comprising the antibiotic compound above is combined with a pharmaceutically acceptable carrier or excipient, including, a diketopiperazine to form a dry powder for oral inhalation, wherein the diketopiperazine is provided in the form of particles. In this embodiment, the diketopiperazine forms crystalline composite particles having a mass median diameter of <10 μm.

In an embodiment, a clofazimine compound in the compositions have antiviral activity against SARS-CoV-2 and have the potential to inhibit viral replication and eliminate viral disease, or eliminate symptoms of viral disease. In in vitro studies, clofazimine is able to reduce or eliminate viral replication by 40% at concentrations of 2.5 μM (or ˜1.2 μg clofazimine/ml). Clofazimine is used to treat bacterial infection and is provided as oral capsules in the treatment of, for example, leprosy and bacterial infections of the lungs, disclosed WO 2020/040818, which disclosure is incorporated by reference as it pertains.

In exemplary embodiments, clofazimine composition, including, CIS and dry powder formulations are used in the treatment of viral infections in general, and those affecting the lungs in particular. Those are infections by coronavirus, influenza, respiratory syncytial virus, Zika, Dengue and the like. In this embodiment, a composition for treating viral infections comprising clofazimine for the therapeutic treatment against SARS-CoV-2 is provided. The composition comprises clofazimine, a derivative of clofazimine, or a salt thereof, or combinations thereof, and a pharmaceutically acceptable carrier and/or excipient thereof for administering to a patient testing positive or diagnosed with the virus. In one embodiment herewith, the clofazimine composition is administered alone, or in combination with other antiviral therapy, including, ribavirin, acyclovir, remdesivir, interferon-beta 1b, lopinavir-ritonavir and the like. In these combination embodiments, the clofazimine composition is administered alone by inhalation and the secondary and/or tertiary therapy can be administered as an inhalable suspension, solution or a dry powder, or by other routes of administration such as oral tablets, oral capsules, injection, intravenously, and the like. In some embodiments, the CIS and dry powder compositions comprising clofazimine are administered in combination with other drugs, including, hydroxychloroquine and invermectin by their prescribed route of administration and dosages.

In some embodiments, clofazimine inhalable compositions, including CIS and dry powders can be used for the treatment of other diseases, including, of leprosy and other bacterial infections, including, in the treatment of nontuberculous mycobacterial diseases. In treatment of bacterial infection using clofazimine capsules, the drug is relatively well tolerated, with the main side effects including yellow skin discoloration and gastrointestinal issues at least in half the patients.

Clofazimine inhalation suspension for inhaled administration through a nebulized form optimizes clofazimine treatment in nontuberculous mycobacteria lung disease (NTM-PD) and tuberculosis (TB). By directly delivering the drug to the airways, CIS is expected to achieve therapeutic lung levels and reduce the incidence of the main side effects (listed above) without impacting therapeutic efficacy. Preclinical studies have evaluated CIS for the treatment of NTM-PD and TB, and a series of toxicology studies have been performed, which have shown that CIS is safe and well tolerated at doses up to ˜3.0 mg/kg (reaching lung levels of ˜10 μg clofazimine/g). The available pharmacokinetic (PK) and pharmacodynamics (PD) studies with a clofazimine inhalation suspension indicate that therapeutic lung concentrations for treatment of COVID-19 disease can be reached through an inhaled loading dose over one or two days and be maintained for up to two weeks.

The benefits and advantages of inhaled clofazimine compositions are numerous and, include, targeted lung dosing (with a fraction of the oral dose), to avoid side effects of high systemic clofazimine levels; protection/prevention of lung infection with virus on lung surface and respiratory cell lining; ability to deliver inhaled therapy at home and away from infectious hospital setting; topical “lung loading” over few days/doses, and prolonged activity with long half-life of clofazimine in the lung.

The clofazimine compositions manufactured in suspension are to be administered to patients with clinical symptoms of COVID-19 disease as an inhaled therapy to be delivered in a home or quarantine setting, as well as under intensive care. The clofazimine compositions have been found to have significant antiviral activity against SARS-CoV-2 in preclinical infection models in vitro, such as VERO-6 cells.

In a particular embodiment, the method for treating pulmonary viral infections comprises, delivering a large dose of clofazimine, a pharmaceutically acceptable derivative, or a salt thereof to the lungs with an aerosol creating a depot of drug in the lungs to release drug over time, so the treatment duration is less than 3 weeks. In one aspect of the method, the composition comprises a suspension administered by a nebulizer or a dry powder. In the embodiment of the dry powder the particle sizes can be from about 1 μm to about 100 μm, from about 1 μm to about 50 μm, from about 2.5 μm to about 25 μm, or from 1 μm to about 10 μm in diameter.

In one embodiment, the method of treatment comprises administering a clofazimine composition to a subject in a dose of about 1 mg to 10 mg of clofazimine daily for a period of 1 week to 14 days. In this embodiment, the method of treatment comprises a clofazimine composition, where the clofazimine, pharmaceutically acceptable derivative, or salt thereof delivered in a single dose of about 3 mg to 8 mg of the active agent in the composition. In certain embodiments, the clofazimine composition is administered in combination with one or more antiviral agents to produce synergistic antiviral activity, including, ribavirin; acyclovir, remdesivir, lopinivir-ritonavir, and interferon-beta 1b. In one embodiment, the inhalable clofazimine composition is administered together with one or more other drugs, including, other antibiotics selected from the group consisting of a daily oral macrolide, inhaled amikacin, and other oral antibiotics, including, amikacin, azithromycin, clarithromycin, tigecycline, cefoxitine, imipenem, pyrazinamide, rifampin, moxifloxacin, levofloxacin and para-amino salicylate, bedaquiline, some of which are administered intravenously or by injection. If more than one combination antibiotic is used, it can be administered by its prescribed dose and route of administration, simultaneously, subsequently, sequentially or after a predetermined period of time following the administration of the inhalable dose. In some embodiments, one or more drugs can be combined with the inhalable clofazimine composition for simultaneous administration.

In another embodiment, a method for the prophylactic treatment of pulmonary viral infections, said method comprising of delivering an effective amount of clofazimine or pharmaceutically acceptable derivative or salt to the patient's lungs with an aerosol, creating a depot of drugs in the lungs to release drug over time, preventing a pulmonary viral infection. With lesser therapeutic amounts of clofazimine administered to a patient, the instant treatment results in the avoidance, or lessening of multiple adverse effects encountered with oral clofazimine therapy. Adverse effects with an oral daily dose of 100 mg to about 300 mg capsule of clofazimine for periods of time which can extend from 30 days to years, include, swelling of lining of gastrointestinal tract, abdominal pain, diarrhea, itchiness, dry skin, changes in skin color, increase in blood sugar, skin sensitivity, and liver toxicity.

In another embodiment, a method of treatment is provided which comprises treating a subject having a viral infection, including, coronavirus, influenza, ebola, or other viral infection which causes lung disease, having the subject inhale a dose of a pharmaceutical composition comprising a dry powder composition comprising particles of clofazimine, a clofazimine derivative, or a salt of clofazimine and a pharmaceutically acceptable carrier and/or excipient using a dry powder inhaler. In this and other embodiments, the pharmaceutically acceptable carrier and/or excipient comprises a compound having the formula:

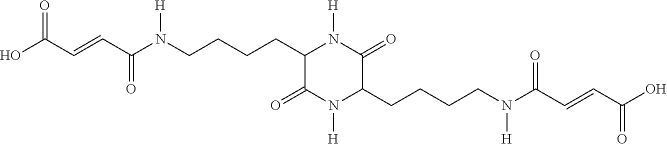 [SMILES: O=C(O)/C=C/C(=O)NCCCCC1NC(=O)C(CCCCNC(=O)/C=C/C(=O)O)NC1=O]

or (E)-4-[4-[(2S,5S)-5-[4-[[(E)-3-carboxyprop-2-enoyl]amino]butyl]-3,6-dioxopiperazin-2-yl]butylamino]-4-oxobut-2-enoic acid, 3,6-bis(N-fumaryl-N(n-butyl)amino)-2,5-diketopiperazine, or a pharmaceutically acceptable salt thereof. The formulation for aerosolization can comprise a crystalline, crystalline composite, or an amorphous dry powder, or combinations thereof.

In an example embodiment, the clofazimine inhalable composition comprises microparticles comprise clofazimine, a derivative of clofazimine or a pharmaceutically acceptable salt thereof, or combinations thereof and a diketopiperazine, wherein the clofazimine, derivative or salt thereof are in an amount of about 1 mg to about 10 mg wt. % in the composition.

In an embodiment, the inhalable dry powder composition can comprise one or more than one carrier and/or excipient, wherein said carrier or excipient is selected from the group consisting of at least one crystalline sugar selected from the group consisting of glucose, arabinose, maltose, saccharose, dextrose, and lactose. In certain embodiments, the carrier or excipient can comprise one or more agents selected from a surfactant, including, polysorbates such as polysorbate 80; phospholipids, including, 1,2-dipalmitoyl-sn-glycero-3-phosphocholine and 1,2-distearoyl-sn-glycero-3-phosphocholine; polymers, and aliphatic amino acids, including, glycine, leucine, isoluceine, and histidine.

In a particular embodiment, the inhalable dry powder composition can comprise carrier or excipient, wherein said carrier or excipient is in a form of finely divided particles having a mass median diameter (MMD) in the range of 0.5 to 10 μm, or 0.5 to 6 μm. In some embodiments, the inhalable dry powder composition comprises a carrier in the form of coarse particles having a mass diameter of 50-500 μm. In other embodiments, the inhalable dry powder composition comprising clofazimine comprises particles having a mass median aerodynamic diameter of less than 5 μm.

In an example embodiment, a pharmaceutical composition for inhalation is provided, comprising a therapeutically effective amount of clofazimine, pharmaceutically acceptable derivative, or salt of clofazimine, and an aqueous liquid carrier selected from water, isotonic saline, buffered saline, and aqueous electrolyte solutions. In this embodiment, the pharmaceutical composition comprising a clofazimine suspension, wherein the clofazimine particles have a mass median diameter of <5 μm, preferably <2 μm. In one embodiment, the pharmaceutical composition comprising clofazimine is solubilized in the form of a micro-emulsion, or nano-emulsion, wherein the emulsion droplets have a mass median diameter (MMD) of less than 1 μm. In this embodiment, the pharmaceutical composition is provided for a nebulization system for use in treating or providing prophylaxis against a pulmonary viral infection, wherein the system comprises the pharmaceutical composition and a nebulizer, and the nebulizer can be selected from the group consisting of a compressed air jet nebulizer, ultrasonic nebulizer, vibrating mesh nebulizer, static mesh nebulizer, or mechanical soft mist inhaler, and wherein the aerosol particles form in use have a mass median aerodynamic diameter (MMAD) of 1 to 5 μm. In one embodiment, the nebulization system further controls the patient's inhalation flow rate either by an electrical or mechanical process and produces an aerosol only when the patient inhales.

In an embodiment, a method of treatment is provided comprising administering to a patient suffering from bacterial lung infections other than viral infections a therapeutically effective amount of a pharmaceutical composition for topical use comprising an antiviral agent, including, clofazimine, and applying said composition to a body surface affected by viral infections in which the pathogen is susceptible to the respective antiviral agent in the formulation.

The instant antiviral compositions can be used also to treat an array of viral infections, for example, the compositions comprising clofazimine can be used for preparation of a various medicament including, for oral, nasal, ophthalmic, pulmonary, parenteral, topical or mucosal application, with pharmaceutically acceptable carriers and excipients.

### EXAMPLES

The following examples are included to demonstrate embodiments of the disclosed particles and compositions for use in methods. It should be appreciated by those of skill in the art that the techniques disclosed in the examples, which follow represent techniques discovered by the inventor to function well in the practice of the present disclosure, and thus can be considered to constitute preferred modes for its practice. However, those of ordinary skill in the art should, in light of the present disclosure, appreciate that many changes can be made in the specific embodiments which are disclosed and still obtain a like or similar result without departing from the scope of the invention.

### Example 1

Clofazimine Inhalable Composition for Dry Powder Inhaler (DPI): An Aerolizer DPI, a dry powder inhaler is provided with the clofazimine drug stored in a capsule. clofazimine is micronized via a jet mill to produce particles with an MMD of <2 μm, then blended with larger lactose particles (MMD>50 μm) to form the clofazimine formulation. The formulation is ˜10% clofazimine by weight. Approximately 250 mg of formulation (25 mg clofazimine) is filled into a capsule. Upon inhalation through the Aerolizer DPI, between 13% and 28% of the dose is deposited in the lungs (Meyer et al, J Aerosol Med, 2004, 17 (1):43-49). Using this inhaler system, a capsule delivers between 3.25 mg and 7 mg of clofazimine to the lungs. One skilled in the art can envision many embodiments that vary slightly in description, but still have the same therapeutic effect of delivering between 3 mg and 8 mg of clofazimine to the lungs with consistency from a single capsule dose. Alternate forms of clofazimine inhalation powder can be manufactured with a pharmaceutically acceptable derivative or salt of clofazimine.

The use of alternative inhalers to deliver the inhalable formulation is accomplished by manufacturing the formulation to be provided for adapting for use with any dry powder inhaler, including, other capsule based devices, blister strip inhalers, reservoir inhalers (metered dose), single use disposable inhalers, and re-usable inhalers. All dry powder inhalers are included by reference including, those disclosed in U.S. Pat. Nos. 8,636,001 and 8,485,180, which disclosures are incorporated herein in their entirety as it pertains.

Alternative particle sizes: Each inhaler has a different resistance to air flow, with higher resistance inhalers resulting in lower inhalation flow rates. Choice of an inhaler with a higher resistance (lower inhalation flow rate) enables the use of larger particle sizes (up to 10 μm) for effective lung delivery. Alternative formulation components: Numerous grades of lactose are available for use in inhalation formulations that vary in size and geometry. Small lactose particles can also be pre-blended to assist in dispersion. Lactose could be replaced with a physiologically acceptable pharmacologically inert solid carrier. Additional excipients such as phospholipids, salts, surfactants or polymers may be added to assist in aerosol dispersion. Alternative formulation forms: Alternatively, clofazimine and excipients can be dissolved in a solvent(s) and spray dried.

### Example 2

PARI eFlow® Nebulizer delivering suspension: An alternative strategy for delivering a therapeutic dose of clofazimine to the lungs uses a nebulizer. Due to the low solubility of clofazimine, clofazimine particles are micronized via jet milling to <2 μm, and mixed with isotonic saline such that the resulting formulation contains between 9 and 20 mg/ml clofazimine. PEG400 or Polysorbate 80 may be added to stabilize the suspension. In this example, approximately 2 mL of formulation is placed in a PARI eFlow® nebulizer. The lung dose from a PARI eFlow® is approximately 25%, so this embodiment results in approximately 4.5 mg of clofazimine deposited in the lungs of the patient.

As with dry powder inhalers, one skilled in the art can envision many embodiments vary slightly in description, but still have the same therapeutic effect of delivering between 3 mg and 80 mg of clofazimine to the lungs. Different nebulizers can be used to deliver the composition. Nebulizers that can deliver the required effective therapeutic dose include compressed air jet nebulizers, ultrasonic nebulizers, vibrating mesh nebulizers, static mesh nebulizers, or mechanical soft mist inhalers. Alternate forms of a clofazimine composition for use with a nebulizer comprises the powder manufactured with a pharmaceutically acceptable derivative or salt of clofazimine. Different concentrations of clofazimine in the formulations can be used depending on the dose to be administered, for example, the concentration of clofazimine can be increased or decreased depending on the patient's requirements. In some embodiments, different excipients such as surfactants can be used to replace PEG400, or be used in combination with PEG400. The compositions for nebulization can also be prepared with different osmolalities, for example, the formulation could be made hypertonic or hypotonic solution or suspension.

To control the inhalation flow rate of the particles in the inhalable composition, the composition is prepared having larger particles per dose, which results in the same total drug amount in the lungs of about 3 mg to 8 mg of clofazimine after administration of the dose being deposited in the lungs.

As stated, drug delivery to the lungs offers many advantages. It is difficult to deliver drugs into the lungs, however, due to problems in trying to get the drugs past natural physical barriers in a uniform volume and weight of the drug.

### Example 3

### Preparation of Crystalline Composite Clofazimine Dry Powders

A 15% clofazimine solution (concentration of clofazimine in this solution could range from 1% clofazimine to 15% clofazimine) was prepared by adding clofazimine (0.20 g) to a 75% acetic acid solution (1.13 g) (concentration of acetic acid solution could range from 75% to 100% acetic acid). The clofazimine solution was added to a microcrystalline particle (XC) suspension (1.31% solids, 175.57 g) suspension of fumaryl diketopiperazine (solids content of the XC suspension could range from 0.5% to 5%). The clofazimine XC suspension was spray dried using a Buchi B-290 spray dryer with the conditions shown in Table 1 to produce an 8% clofazimine XC powder.

### Preparation of Crystalline Clofazimine Dry Powders

A 15% clofazimine solution (concentration of clofazimine in this solution could range from 1% clofazimine to 15% clofazimine) was prepared by adding clofazimine (0.20 g) to a 75% acetic acid solution (1.13 g) (concentration of acetic acid solution could range from 75% to 100% acetic acid). The clofazimine solution was added to a suspension of 3,6-bis(N-fumaryl-4-aminobutyl)-2,5-diketopiperazine pre-formed particles (T suspension; 11.04% solids, 20.83 g) (solids content of the T suspension could range from 0.5% to 20%). The clofazimine T suspensions were then dried by spray drying to produce an 8% clofazimine T powder. Powders were spray dried using a Buchi B-290 spray dryer with conditions shown in Table 1.

TABLE 1
Clofazimine Powder Spray Drying Conditions

| Spray Dryer Parameter | Set Point | |
|---|---|---|
| Inlet Temperature | 180° | C. |
| Aspirator Pump Speed | 90% | |
| Feed Pump Speed | 25% | |
| Nitrogen Flow | 60 | mm |

### Powder Testing

Powders were evaluated for aerodynamic particle size distribution using an Andersen Cascade impactor (ACI). Powders were discharged through the ACI from Gen 2C cartridges (10 mg cartridge fills) at 4 kPa. Data for the clofazimine powders prepared to date is shown in Table 2.

TABLE 2
Clofazimine Powder Data

ACI Data

| Powder | Drying | Yield | Avg. CE | RF |
|---|---|---|---|---|
| Description | Technique | (%) | (%) | (%) |
| 8% Clofazimine | Spray | 54.4 | 90.9 | 49.6 |
| XC Powder | Drying | | | |
| 8% Clofazimine | Spray | 79.6 | 83.2 | 38.4 |
| T Powder | Dried | | | |

As can be seen in Table 2, the process product yield was greater than 50% for both sprayed dried dry powders and the average powder delivered from the delivery system was greater than 80% as assessed by cartridge emptying (CE) measurements.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a” and “an” and “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed. No language in the specification should be construed as indicating any non-claimed element essential to the practice of the invention.

The use of the term “or” in the claims is used to mean “and/or” unless explicitly indicated to refer to alternatives only or the alternatives are mutually exclusive, although the disclosure supports a definition that refers to only alternatives and “and/or.”

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Of course, variations on those preferred embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Furthermore, numerous references have been made to patents and printed publications throughout this specification. Each of the above cited references and printed publications are herein individually incorporated by reference in their entirety.

Further, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. An inhalable pharmaceutical composition comprising an antiviral agent selected from clofazimine, a pharmaceutically acceptable derivative of clofazimine, a clofazimine salt, or a polymorph of clofazimine, or combination thereof, and a pharmaceutically acceptable carrier and/or excipient; wherein the antiviral agent is in the amount of 1 mg to 20 mg wt % dose in the composition.

2. The inhalable composition of claim 1, wherein the antiviral agent and the pharmaceutically acceptable carrier and/or excipient are formulated for oral inhalation.

3. The inhalable composition of claim 2, wherein the antiviral agent and the pharmaceutically acceptable carrier and/or excipient are formulated as a suspension for pulmonary delivery by nebulization and the pharmaceutically acceptable carrier and/or excipient is and aqueous liquid carrier selected from water, isotonic saline, buffered saline, or aqueous electrolyte solutions.

4. The inhalable composition of claim 2, wherein the antiviral agent and the pharmaceutically acceptable carrier and/or excipient are formulated as a dry powder for oral inhalation.

5. The inhalable composition of claim, wherein the antiviral agent is an orthorhombic crystal polymorph of clofazimine.

6. The inhalable composition of claim 4, wherein the pharmaceutically acceptable carrier and/or excipient is a diketopiperazine.

7. The inhalable composition of claim 4, wherein the diketopiperazine is of the formula (E)-4-[4-[(2S,5S)-5-[4-[[(E)-3-carboxyprop-2-enoyl]amino]butyl]-3,6-dioxopiperazin-2-yl]butylamino]-4-oxobut-2-enoic acid.

8. The inhalable pharmaceutical composition of claims 1-7, characterized in that the composition is for use in the manufacture of a medicament for the treatment of a viral infections.

9. The inhalable pharmaceutical composition of claims 1-7 for use in the treatment of SARS-CoV-2 pulmonary viral infection.

10. A method for treating pulmonary a viral infection, said method comprising delivering a therapeutically effective orally inhalable dose of a pharmaceutical composition comprising clofazimine, pharmaceutically acceptable derivative of clofazimine, a polymorph of clofazimine, or a clofazimine salt, and a pharmaceutically acceptable carrier and/or excipient to a patient's lungs with an aerosol from a dry powder inhaler or a nebulized suspension.

11. The method of claim 10, wherein the therapeutically effective orally inhalable dose is from about 1 mg to 20 mg wt % of the clofazimine, pharmaceutically acceptable derivative of clofazimine, a polymorph of clofazimine, or a clofazimine salt.

12. The method of claim 11, where the viral infection is coronavirus, influenza, ebola or other viral infection affecting the lung, or a combination thereof, wherein the viral infection is further treated with one or more antiviral agents.

13. The method of claim 11, wherein between 3 and 8 mg of clofazimine is delivered to the lungs.

14. The method of claim 11, wherein a 3 mg to 8 mg single daily dose for 7 days of Clofazimine, a pharmaceutically acceptable derivative of clofazimine, a polymorph of clofazimine, or a clofazimine salt is administered to the patient to create a depot of drug in the lungs to release drug over time and treatment duration is less than 3 weeks.

15. An inhalable dry powder 4, wherein said carrier is selected from the group consisting of at least one crystalline sugar selected from the group consisting of glucose, arabinose, maltose, saccharose, dextrose, and lactose.

16. The inhalable dry powder of claim 15, wherein said carrier is in a form of finely divided particles having a mass median diameter (MMD) in the range of 0.5 to 10 μm.

17. A pharmaceutical composition comprising a therapeutically effective amount of clofazimine, pharmaceutically acceptable derivative, a polymorph of clofazimine, or salt of clofazimine and an aqueous liquid carrier selected from water, isotonic saline, buffered saline, and aqueous electrolyte solutions.

18. The pharmaceutical composition of claim 17, further comprising particles of the clofazimine, pharmaceutically acceptable derivative, a polymorph or clofazimine, or salt of clofazimine in a suspension, wherein the clofazimine particles have a mass median diameter of <5 μm, preferably <2 μm.

19. The pharmaceutical composition of claim 17, wherein the clofazimine, pharmaceutically acceptable derivative, a polymorph of clofazimine, or clofazimine salt is solubilized in the form of a micro- or nano-emulsion, wherein the emulsion droplets have a mass median diameter (MMD) of less than 1 μm.

20. An inhalation system for use in the treatment or prophylaxis of a pulmonary viral infection wherein the system comprises a pharmaceutical composition according to claim 17 and a nebulizer selected from a compressed air jet nebulizer, ultrasonic nebulizer, vibrating mesh nebulizer, static mesh nebulizer, or mechanical soft mist inhaler, and wherein the aerosol particles produced have a mass median aerodynamic diameter (MMAD) of 1 to 5 μm.
